(19) United States
(12) Patent Application Publication
(10) Pub. No.: US 2018/0028543 A1
(43) Pub. Date: Feb. 1, 2018

(54) HYPOLIPIDEMIC COMPOSITION AND USE THEREOF

(71) Applicants: Honggui WAN, Jiangsu (CN); Maoxing YUE, Beijing (CN)

(72) Inventors: Honggui WAN, Nanjing (CN); Maoxing YUE, Beijing (CN)

(21) Appl. No.: 15/551,031

(22) PCT Filed: Jul. 2, 2015
(86) PCT No.: PCT/CN2015/083178

§ 371 Date: Aug. 14, 2017

(30) Foreign Application Priority Data
Jul. 3, 2014 (CN) 201410315242.X

(51) Int. Cl. A61K 31/675; A61K 31/198; A61K 31/375; A61K 31/194; A21D 2/30; A61K 31/519; A21D 2/24; A23L 33/175; A23L 33/15; A61K 31/197; A61K 31/4188

(52) U.S. Cl. CPC A61K 31/675; A61K 31/197; A61K 31/198; A61K 31/375; A61K 31/194; A61K 31/4188; A61K 31/519; A21D 2/245; A23L 33/175; A23L 33/15; A21D 2/30; A23V 2002/00

(57) ABSTRACT

A composition has ornithine, aspartic acid and vitamin B6, and it is used in preparation of hypolipidemic drugs, health-care products, foods or food additives.

### BACKGROUND

### Technical Field

The present invention relates to the technical fields of health-care products, foods and medicine, in particular to a hypolipidemic composition and use thereof.

### Related Art

Hyperlipidemia means that one or more of lipids in plasma are higher than normal due to lipometabolism or functioning abnormality. It is a systemic disease with the blood total cholesterol (TC) and (or) triglyceride (or) being too high or high-density lipoprotein cholesterol (HDL-C) being too low, which is called dyslipidemia in modern medicine, and often called hyperlipoproteinemia. Most of hyperlipidemia has no any symptom in an initial stage. If not promptly corrected, the hyperlipidemia will generate progressive systemic damage to the body. It will accelerate systemic atherosclerosis, and then cause kidney failure. The current data research shows that the hyperlipidemia can induce the following diseases: coronary heart disease, myocardial infarction, sudden cardiac death, cerebral stroke, hypertension, diabetes, fatty liver, cirrhosis, cholelithiasis, pancreatitis, hyperuricemia and the like.

Amino acids are the most basic substances that constitute the organism protein and are related to life activities, are the basic unit constituting protein molecules in an organism, and are closely related to the life activities of the organism. The amino acids have a special physiological function in antibodies, and are one of indispensable nutrients in the organism. The amino acids can play the following roles through metabolism in the human body: \textcircled{1} synthesizing tissue proteins; \textcircled{2} changing into acids, hormones, antibodies, ceatine and other ammonia-containing substances; \textcircled{3} converting into carbohydrates and fats; \textcircled{4} oxidating into carbon dioxide, water and urea, and generating energy. Ornithine, which is a non-protein amino acid, can activate key enzymes of urea synthesis—ornithine carbamyl transferase and carbamyl phosphate synthetase, a reaction substrate ornithine can be provided, the synthesis of urea is accelerated through liver ornithine cycle, and the ammonia metabolism is promoted, so that the detoxification of blood ammonia can be realized. Aspartic acid can be directly involved in the urea cycle and involved in the triphosphate cycle and the synthesis of nucleic acids in liver cells firstly, and is conducive to repairing damaged liver cells. In addition, the aspartic acid has an indirect promotion effect on the process of metabolism of tricarboxylic acid in liver cells, and provides intermediate products of energy metabolism to promote the energy generation of liver cells, so that various functions of the damaged liver cells can be rapidly restored.

B vitamins are water-soluble vitamins, including vitamin B1, vitamin B2, vitamin B6, vitamin B12, niacin, pantothenic acid, folic acid and the like, and are indispensable substances that promote in vivo metabolism, and convert sugar, fat, protein and the like into heat. They have a synergistic effect, regulate metabolism, enhance the immune system and promote cell growth and division. B6 may be the most important one of all B vitamins. The body's muscles contain 70% to 80% of B6 in the whole body. B6 plays a key role in protein, lipid and carbohydrate metabolism. Therefore, the loss of a large number of B6 will cause amino acid metabolic disorders. Vitamin B6 is an important coenzyme for amino acid metabolism and synthesis, participates in the metabolism and other physiological processes of unsaturated fatty acids, is a coenzyme of many important enzyme systems in the organism, and is a nutrient necessary for animal normal development and bacteria and yeast reproduction. In addition, vitamin B6 is also a natural diuretic, diuresis plays a detoxification role, and 380 ml or so of urine can be produced by intravenously infusing 5 g of vitamin B6. The vitamin B6 is a coenzyme of the human body's amino acid metabolism, a neurotransmittery-aminobutyric acid (GABA) and glutamate (Glu). It is known that the vitamin B6 needs to be involved in more than 60 kinds of enzymes in the liver, and plays a very important role in promoting the normal enzyme metabolism of the organism, and the vitamin B6 has a short half life in vivo and is rapidly excreted.

As we all know, the liver plays an important role in lipid digestion, absorption, decomposition, synthesis and transportation and other metabolic processes. Lipid modification, synthesis, decomposition, ketone body formation, lipoprotein metabolism, etc. are carried out in the liver. Liver can secrete bile, and bile salts in bile can promote the digestion and absorption of lipids. Triglycerides absorbed from the digestive tract are not assimilated in the liver cells, and are transported to the adipose tissue to be stored; when a person feels hungry, the stored fat is mobilized to the liver and other tissues for catabolism. In the liver, β-oxidation of fatty acids is particularly active, so that a large number of acetyl-CoA(HMG-CoA) is generated, and the HMG-CoA is rapidly decomposed into the ketone body. And lipid transport and transformation are closely related to the liver function. For example, the very low density lipoprotein (VLDL) in plasma is mainly synthesized in the liver, is converted into low-density lipoprotein (LDL) in the plasma, and releases part of high-density lipoprotein (HDL), while an important enzyme—lecithin cholesterol transacylase (LCAT) catalyzing these changes is also synthesized in the liver cells. If the above metabolic reactions are hampered, the lipid content in the liver will change; when the fat is excessively accumulated in the liver, the fatty liver will be caused. Chronic fatty liver can cause liver cell fibrosis, resulting in liver cirrhosis, and damaging the normal function of liver cells. The main occurrence principle of fatty liver is the excessive fat and fatty acids input into the liver, and the anabolism disorders of plasma lipoprotein in the liver.

In the modern society, with the improvement of living standards and the changes in dietary structure of people, the prevalence of hyperlipidemia is on the rise around the world. So far there are many drugs for treatment of hyperlipidemia, and most of them are chemically synthesized chemicals or Chinese herbal compounds formed by some traditional Chinese medicines. Chemical synthetic drugs have a small amount of harmful substances because of its production process, and have toxicity after long-term use, and the late effect is not very satisfactory; the Chinese herbal compound preparation needs to be used for a long time because of its moderate efficacy. Therefore, it is very necessary to develop novel effective hypolipidemic drugs or health-care products to reduce the health hazard of the blood lipid concentration to the human body.

### SUMMARY

The object of the present invention is to provide a hypolipidemic composition for the above-mentioned technical problems. The hypolipidemic composition is a composition of compound amino acids and vitamins designed for patients suffering from hyperlipidemia, and can effectively reduce blood lipid concentration and awaken the ability of lowering blood lipid of patient bodies.

Another object of the present invention is to provide use of the above-mentioned hypolipidemic composition.

The objects of the present invention are achieved by the following technical solutions:

the hypolipidemic composition comprises ornithine, aspartic acid and vitamin b6.

The composition comprises the following components by a weight ratio: ornithine:aspartic acid:VB6=1:0.1-1.2:0.2-1.5, preferably ornithine:asparticacid:VB6=1:0.3-0.7:0.4-0.8, and further preferably ornithine:asparticacid:VB6=1:0.5:0.6.

The composition also comprises one or more of the following auxiliary components: vitamin C, citric acid, pantothenic acid, biotin and folic acid,

wherein the amounts of the auxiliary components are as follows by a weight ratio:

ornithine:VC=1:0.05-0.2, preferably, ornithine:VC=1:0.2-0.8, and further preferably, ornithine:VC=1:0.5;

ornithine:citric acid=1:0.001 to 0.05, preferably, ornithine:citric acid=1:0.001 to 0.03, and further preferably, ornithine:citric acid=1:0.01;

ornithine:pantothenic acid=1:0.00001-0.001, preferably, ornithine:pantothenic acid=1:0.00005-0.0008, and further preferably, ornithine:pantothenic acid=1:0.0005;

ornithine:biotin=1:0.000001-0.0005, preferably, ornithine:biotin=1:0.000001-0.0001, and further preferably ornithine:biotin=1:0.000003;

ornithine:folic acid=1:0.000001-0.0005, preferably, ornithine:folic acid=1:0.000003-0.0001, and further preferably, ornithine:folic acid=1:0.00005.

In the composition, the ornithine refers to L-ornithine, D-ornithine or DL-ornithine; and the aspartic acid refers to L-aspartic acid, D-aspartic acid or DL-aspartic acid.

The dosage form of the composition as a health-care product or a drug is any one of pharmaceutically acceptable dosage forms. The preferable dosage forms are granules, tablets, capsules, powders, pills, powders, medicinal liquors, ointments, pellets or granules, membranes, oral solutions and injections.

The composition is applied in the preparation of hypolipidemic health-care products, drugs, foods or food additives.

The food additives can be added and applied to various kinds of foods, for example, the food additives can be used as additives for baking pastries, drinks, butter dairy products and the like.

The technical solutions of the invention comprehensively utilize the effect characteristics of the ornithine, the aspartic acid, the vitamin B6 and other substances; when the ornithine, the aspartic acid and the vitamin B6 are used in combination, the ornithine used as a reaction substrate of the urea cycle rapidly activates the urea cycle in the liver cells, so that the enzyme metabolism in liver is gradually restored, and thus the vitality of the liver can be restored to play a liver protecting role. Aspartic acid can be directly involved in the urea cycle and involved in the triphosphate cycle and the synthesis of nucleic acids in liver cells firstly, and is conducive to repairing damaged liver cells. In addition, the aspartic acid has an indirect promotion effect on the process of cycle metabolism of tricarboxylic acid in liver cells, and provides intermediate products of energy metabolism to promote the energy generation of liver cells, so that various functions of the damaged liver cells can be rapidly restored. Vitamin B6 is an important coenzyme for amino acid metabolism and synthesis, participates in the metabolism and other physiological processes of unsaturated fatty acids, is a coenzyme of many important enzyme systems in the body, and is a nutrient necessary for animal normal development and bacteria and yeast reproduction. And the vitamin B6 is also a natural diuretic, and diuresis plays a detoxification role. Therefore, the ornithine, the aspartate and the vitamin B6 used in combination can effectively repair the damaged liver cells, restore the liver function, and wake up the body's ability to reduce the blood lipid. The three are used in combination to produce phosphatidylcholine to help the emulsification of fat; the liver can also promote the synthesis of bile salts, and digest a large number of triglycerides. The liver is not only an important organ for synthesizing cholesterol in the body, but also cholesterol transported into the liver by the high-density lipoprotein can also be converted in the liver cells and excreted. Part of cholesterol directly used as a bile component together with a bile salt enters the intestine through a bile duct, and the metabolic waste can be smoothly excreted under the action of the vitamin B6 at this time.

The composition provided by the invention has the advantages that:

the curative effect is significant on hyperlipidemia; compared with the existing chemical synthetic drug and traditional Chinese herb compound hypolipidemic method, the composition prepared by the invention not only has less side effect, less recurrence of the disease, and short treatment cycle, but also provides nutritional support for the patients, effectively restores the hypolipidemic function of the body, and promotes enzyme metabolism to achieve the purpose of treating both symptoms and root causes.

The components adopted by the composition provided by the invention can be all derived from food, have high safety, and have no toxic and side effect after being eaten for a long time, but the content ratio of the components is inconsistent with that in food. It is found through experiments that the composition can play a hypolipidemic role while the general food has no such effect.

### DETAILED DESCRIPTION

Hereinafter, the present invention will be further described in detail in conjunction with the examples, but the present invention is not limited to the following examples.

### Example 1: Preparation of Hypolipidemic Sandwich Biscuit by Using Hypolipidemic Composition as Food Additive

The preparation process comprises the following steps:

1. blending wheat flour, eggs, a sweetener, baking soda, vegetable oil and water into dough according to a weight ratio, putting in a refrigerator for cold storage, resting for 1 hour, then taking out, rolling into 2 to 3 mm slices, pressing into a shape using a biscuit mold, one being hollow and the other being solid, and preparing into a biscuit ectoderm;

2. putting in an oven for baking, and cooling for standby use;

3. weighing an appropriate amount of jam, the total weight of the jam, L-ornithine, L-aspartic acid and vitamin B6 being 100 parts, evenly mixing the following raw materials in parts by weight: 3 parts of the L-ornithine, 2 parts of the L-aspartic acid, 3 parts of the vitamins B6 and 92 parts of the jam, preparing a food additive, and then smearing the food additive on a hollow part of the biscuit.

### Example 2: Preparation of Healthcare Oral Solution

A hypolipidemic health-care oral solution was prepared by the following steps: weighing the following raw materials by weight: 20 g of L-ornithine, 5 g of L-aspartic acid, 25 g of vitamin B6, 15 g of vitamin C, 5 g of xylitol and 20 ml of water, mixing evenly, canning a mixture into a 5-50 mL bottle, sealing, and sterilizing at a temperature of 110° C. for 20 min.

### Example 3: Preparation of Pharmaceutical Composition

A pharmaceutical composition capsule preparation comprises the following components: 70 g of L-ornithine, 60 g of L-aspartic acid, 70 g of vitamin B6, an appropriate amount of 1% polyvinylpyrrolidone ethanol solution and 2 g of magnesium stearate, and 800 capsules were prepared in total.

The preparation method comprises the following steps: weighing the L-ornithine, the L-aspartic acid and the vitamin B6, mixing evenly, preparing a soft material with 40 mL of the 1% polyvinylpyrrolidone ethanol solution, granulating with a 20-mesh sieve, drying until the moisture does not exceed 3%, adding 2 g of the magnesium stearate, mixing evenly, granulating with the 20-mesh sieve, sub-packaging, polishing and packaging, thus obtaining a capsule preparation through the above process.

### Example 4: Preparation of Pharmaceutical Composition

A pharmaceutical composition tablet comprises the following components: 70 g of L-ornithine, 60 g of L-aspartic acid, 70 g of vitamin B6, an appropriate amount of 1% polyvinylpyrrolidone ethanol solution and 1 g of magnesium stearate, and 1000 tablets were prepared in total.

The preparation method comprises the following steps: weighing the L-ornithine, the L-aspartic acid and the vitamin B6, mixing evenly, preparing a soft material with 20 mL of the 1% polyvinylpyrrolidone ethanol solution, granulating with a 20-mesh sieve, drying, adding 1 g of magnesium stearate, mixing evenly, granulating with the 20-mesh sieve, tabletting, polishing and packaging, thus obtaining a composition tablet through the above process.

### Example 5: Preparation of Pharmaceutical Composition

A pharmaceutical composition comprises 250 mL of a mixed substance injection containing 6 g of L-ornithine, 4 g of L-aspartic acid, 6 g of vitamin B6, 3.5 g of vitamin C, and 0.1 mg of biotin, and was added into 250 mL of a 5% glucose and sodium chloride injection (0.9% sodium chloride injection used for diabetic patients).

### Example 6

A pharmaceutical composition granule comprises the following components: 70 g of L-ornithine, 60 g of L-aspartic acid, 70 g of vitamin B6, 0.5 g of citric acid, 5 mg of pantothenic acid, 0.2 mg of biotin, 0.3 mg of folic acid and 15 g of vitamin C. An appropriate amount of 20% ethanol solution was used as a wetting agent, so that the granule was prepared,

The preparation method comprises the following steps: weighing the above-mentioned components, mixing evenly, adding 40 ml of 20% ethanol solution, preparing a soft material, preparing the granule by an extrusion-spheronization method, and drying until the moisture is not more than 3%.

### Example 7

A pharmaceutical composition granule comprises the following components: 70 g of L-ornithine, 60 g of D-aspartic acid, 70 g of vitamin B6, 0.5 g of citric acid, 0.2 mg of biotin and 20 g of vitamin C. An appropriate amount of 20% ethanol solution was used as a wetting agent, so that the granule was prepared.

The preparation method comprises the following steps: weighing the above-mentioned components, mixing evenly, adding 40 ml of 20% ethanol solution, preparing a soft material, preparing the granule by an extrusion-spheronization method, and drying until the moisture is not more than 3%.

### Example 8

A pharmaceutical composition granule comprises the following components: 70 g of DL-ornithine, 50 g of L-aspartic acid, 60 g of vitamin B6, 30 mg of pantothenic acid, 0.5 mg of biotin and 20 g of vitamin C. An appropriate amount of 20% ethanol solution was used as a wetting agent, so that the granule was prepared.

The preparation method comprises the following steps: weighing the above-mentioned components, mixing evenly, adding 40 ml of 20% ethanol solution, preparing a soft material, preparing the granule by an extrusion-spheronization method, and drying until the moisture is not more than 3%.

### Example 9

A pharmaceutical composition granule comprises the following components: 70 g of L-ornithine, 50 g of DL-aspartic acid, 60 g of vitamin B6, 30 mg of pantothenic acid, 3 mg of folic acid and 5 g of vitamin C. An appropriate amount of 20% ethanol solution was used as a wetting agent, so that the granule was prepared.

The preparation method comprises the following steps: weighing the above-mentioned components, mixing evenly, adding 40 ml of 20% ethanol solution, preparing a soft material, preparing the granule by an extrusion-spheronization method, and drying until the moisture is not more than 3%.

### Example 10

A pharmaceutical composition granule comprises the following components: 70 g of L-ornithine, 50 g of DL-aspartic acid, 35 g of vitamin B6 and 45 g of vitamin C. An appropriate amount of 20% ethanol solution was used as a wetting agent, so that the granule was prepared.

The preparation method comprises the following steps: weighing the above-mentioned components, mixing evenly, adding 40 ml of 20% ethanol solution, preparing a soft material, preparing the granule by an extrusion-spheronization method, and drying until the moisture is not more than 3%.

### Example 11: Hypolipidemic Animal Experiment of Composition Provided by the Invention

1. Experimental materials: 150 healthy SD male and female rats in clean grade, with body weight of (150.2±8.9) g.

2. Feed: general feed, high-fat feed formula: 72.7% of basic feed, 2% of cholesterol, 5% of egg yolk powder, 10% of lard, 0.2% of propylthiouracil, 10% of sucrose and 0.1% of sodium cholate.

3. Grouping and administration of animals: the purchased experimental rats were adaptively fed for 1 week in a lab, original numbers were assigned according to the weighing order, and the rats were randomly divided into a blank control group, a model control group, a pharmaceutical composition (respectively prepared as in Examples 3, 6, 8 and 10, and added with 50 ml of water per 10 g to be dissolved to obtain a solution) low-dose group (5 ml/kg·d), a middle-dose group (10 ml/kg-d), a high-dose group (15 ml/kg·d), a positive drug (simvastatin) control group (1.5 mg/kg, volume: 10 ml/kg) by looking up a random number table, and there were 10 rats in each group. From the day of experiment, the blank control group and the model control group were intragastrically administered with normal saline (volume: 10 ml/kg), the administration group was intragastrically administered with corresponding drugs once a day, the body weight was weighed once a week, and the dosage was adjusted according to the body weight. The blank control group was fed with the normal feed, the other groups was fed with high-fat feed. During the experiment, free feeding, free drinking, continuous molding and drug administration for prevention were carried out for 4 weeks.

4. Observation indexes: after the last administration, the rats fasted for 12 h without fasting water. The rats were anesthetized with 3% pentobarbital sodium, blood was collected from the abdominal aorta, serum was centrifugally separated, and the serum total cholesterol (TC), triglyceride (TG), high-density lipoprotein cholesterol (HDL-C), low-density lipoprotein cholesterol (LDL-C), alanine aminotransferase (ALT) and aspartate aminotransferase (AST) values were measured.

5. Results

a) Effect of the Composition Provided by the Invention on Blood Lipid Levels in Rats

As shown in Table 1, compared with the normal group, the levels of TC, TG and LDL-C in the serum of the rats in the model group were significantly increased (P<0.05), showing typical lipid metabolism disorder, thus indicating that an experimental hyperlipidemia model was successfully replicated.

Compared with the model group, the contents of TC, TG and LDL-C in the serum in the treatment group were significantly decreased (P<0.05), and the HDL-C content of the serum was significantly increased (P<0.01) in a dose-dependent manner.

TABLE 1
Changes of blood lipids of rats in various groups(X̅ ± S, n = 10)

| | | TC | TG | HDL-C | LDL-C |
|---|---|---|---|---|---|
| Groups | | (mmol/L) | (mmol/L) | (mmol/L) | (mmol/L) |
| Normal control group | | 1.63 ± 0.53 | 0.71 ± 0.42 | 1.36 ± 0.25 | 0.26 ± 0.27 |
| Model control group | | 12.34 ± 3.17 | 1.20 ± 0.45 | 0.94 ± 0.36 | 5.67 ± 3.37 |
| Example 3: | Low-dose group | 6.19 ± 1.70 | 0.70 ± 0.27 | 1.56 ± 0.15 | 2.40 ± 1.87 |
| Drug | Middle-dose group | 5.91 ± 3.01 | 0.67 ± 0.20 | 1.64 ± 0.10 | 1.88 ± 1.49 |
| | High-dose group | 1.65 ± 0.21 | 0.57 ± 0.19 | 1.66 ± 0.69 | 0.99 ± 0.83 |
| Example 6: | Low-dose group | 6.17 ± 1.62 | 0.69 ± 0.22 | 1.53 ± 0.11 | 2.33 ± 1.76 |
| Drug | Middle-dose group | 4.67 ± 3.18 | 0.60 ± 0.15 | 1.62 ± 0.08 | 1.85 ± 1.40 |
| | High-dose group | 1.61 ± 0.21 | 0.53 ± 0.16 | 1.65 ± 0.61 | 0.97 ± 0.71 |
| Example 8: | Low-dose group | 6.05 ± 1.71 | 0.60 ± 0.24 | 1.46 ± 0.13 | 2.28 ± 1.80 |
| Drug | Middle-dose group | 4.51 ± 3.21 | 0.52 ± 0.19 | 1.55 ± 0.10 | 1.80 ± 1.46 |
| | High-dose group | 1.52 ± 0.23 | 0.43 ± 0.18 | 1.59 ± 0.61 | 0.95 ± 0.73 |
| Example 10: | Low-dose group | 6.10 ± 1.69 | 0.65 ± 0.27 | 1.49 ± 0.10 | 2.29 ± 1.83 |
| Drug | Middle-dose group | 4.58 ± 3.25 | 0.58 ± 0.17 | 1.59 ± 0.15 | 1.83 ± 1.50 |
| | High-dose group | 1.56 ± 0.19 | 0.50 ± 0.16 | 1.63 ± 0.65 | 0.96 ± 0.76 |

| | | | | |
|---|---|---|---|---|
| Positive drug control group | 8.42 ± 4.79 | 0.49 ± 0.18 | 1.23 ± 0.17 | 1.68 ± 1.91 |

b) Effect of the Composition Provided by the Invention on Liver Function of Rats

The liver function could be reflected by ALT and AST in the serum. The levels of ALT and AST in the high-fat feed group were significantly higher than those in the basic feed group (P<0.01), indicating that there was a certain damage to the liver. The levels of ALT and AST in the serum in each administration group were lower than those in the model control group, and there was a significant difference between the middle-dose group, the high-dose group and the model control group (P<0.01), indicating that the composition provided by the invention has a significant effect on protection of liver function, and a significant protecting effect on the organism.

TABLE 2
Changes of liver function of rats in each group (X̅ ± S, n = 10)

| | | Dose | ALT | AST |
|---|---|---|---|---|
| Groups | | (ml/kg-d) | (U/L) | (U/L) |
| Normal control group | | — | 40.39 ± 6.80 | 19.74 ± 3.16 |
| Model control group | | — | 55.44 ± 2.88 | 31.51 ± 5.68 |
| Example 3 | Low-dose | 5 | 48.19 ± 5.52 | 26.71 ± 1.66 |
| Drug | group | | | |
| | Middle-dose | 10 | 44.33 ± 3.94 | 20.88 ± 7.01 |
| | group | | | |
| | High-dose | 15 | 42.55 ± 4.49 | 18.39 ± 5.70 |
| | group | | | |
| Example 6 | Low-dose | 5 | 48.16 ± 5.57 | 26.66 ± 1.64 |
| Drug | group | | | |
| | Middle-dose | 10 | 44.27 ± 3.91 | 20.81 ± 7.11 |
| | group | | | |
| | High-dose | 15 | 42.46 ± 4.47 | 18.37 ± 5.77 |
| | group | | | |
| Example 8 | Low-dose | 5 | 48.05 ± 5.59 | 26.63 ± 1.60 |
| Drug | group | | | |
| | Middle-dose | 10 | 44.19 ± 3.95 | 20.77 ± 7.15 |
| | group | | | |
| | High-dose | 15 | 42.43 ± 4.40 | 18.34 ± 5.79 |
| | group | | | |
| Example 10 | Low-dose | 5 | 48.18 ± 5.57 | 26.67 ± 1.65 |
| Drug | group | | | |
| | Middle-dose | 10 | 44.22 ± 3.99 | 20.84 ± 7.11 |
| | group | | | |
| | High-dose | 15 | 42.49 ± 4.47 | 18.38 ± 5.70 |
| | group | | | |

| | | | |
|---|---|---|---|
| Positive drug control group | 1.5 mg/Kg-d | 45.85 ± 5.21 | 22.32 ± 7.56 |

The contents of TC, TG and LDL-C were significantly decreased while the content of HDL-C was increased after the hyperlipidemia rats were drenched with the composition provided by the present invention; and the increase in ALT and AST in the serum caused by the high-fat feed was inhibited. It was indicated that the composition provided by the invention promoted fatty acid metabolism, improved intrahepatic lipid metabolism, protected liver cells and regulated lipids and protected liver, and was especially suitable for hyperlipidemia patients with high risk caused by liver function damage. Pharmacological experiments proved that the ornithine, the aspartic acid and the vitamin B6 had a synergistic effect, and could enhance the efficacy each other.

### Example 12: Effect of Composition Provided by the Invention in Clinical Treatment

1. Case Selection

Patients with more than 5.7 mmol/L of serum total cholesterol (TC) and (or) more than 1.7 mmol/L of triglyceride (TG) were selected as observation objects, and there were 24 cases in total, including 14 males and 10 females, aged 26 to 69 years.

2. Diagnostic Criteria

Referring to “Internal Medicine” published by People's Medical Publishing House in 2010, the diagnostic criteria of hyperlipidemia was as follows: serum total cholesterol (TC) was more than or equal to 5.72 mmol/L, triglyceride (TG) was more than or equal to 1.70 mmol/L, low-density lipoprotein cholesterol (LDL-C) was more than or equal to 3.10 mmol/L, and high-density lipoprotein (HDL-C) was less than or equal to 1.04 mmol/L for males, and less than or equal to 1.17 mmol/L for females.

3. Treatment Method

All patients were prohibited from drinking alcohol and eating excessive lipid food at dinner on the day before blood drawing, and TC, TG, HDL-C, and LDL-C were measured by drawing venous blood after the patients fasted for more than 12 hours. Then, the composition provided by the present invention (prepared as in Example 3) was administered three times a day for four continuous weeks, two pills were taken once, each pill contained about 0.25 g of the composition provided by the present invention, and spicy food, especially alcohol was restrained during treatment.

4. Efficacy Evaluation Standards

Markedly effective: After treatment, the blood lipid was detected to conform to any one of the following standards: TC was decreased by more than or equal to 20%, or TG was decreased by more than or equal to 40%, or HDL-C was increased by more than or equal to 0.26 mmol/L, or HDL-C/LDL-C was decreased by more than or equal to 20%, or LDL-C was decreased by more than or equal to 30%.

Effective: the blood lipid was detected to conform to any one of the following standards: TC was decreased by more than or equal to 10% and less than 20%, or TG was decreased by more than or equal to 20% and less than 40%, or HDL-C was increased by more than or equal to 0.14 mmol/L and less than 0.26 mmol/L, or HDL-C/LDL-C was decreased by more than or equal to 10% and less than 20%, or LDL-C was decreased by more than or equal to 20%.

Ineffective: the blood lipid was obviously improved or the improvement on the blood lipid did not conform to the standards before and after treatment.

5. Treatment Effects

After 24 cases of patients were treated with the composition provided by the invention for two weeks, the composition was markedly effective in 8 cases, effective in 11 cases, and ineffective in 5 cases, and TC and TG were decreased to different degrees in they ineffective cases. After the composition provided by the invention was continuously administered for two weeks, the composition was markedly effective in 14 cases, effective in 9 cases, and ineffective in 1 case, the inefficiency was 4.2%, and the total efficiency was 95.8%.

6. Description of Specific Cases

(1) Gao, male, 33 years old, hyperlipidemia. Before treatment, the total cholesterol (TC) was 6.31 mmol/L and the triglyceride (TG) was 2.16 mmol/L. The total cholesterol (TC) was decreased to 4.92 mmol/L and the triglyceride (TG) was decreased to 0.99 mmol/L two weeks after administration of the composition provided by the present invention.

(2) Xu, female, 69 years old, hyperlipidemia. Before treatment, the total cholesterol (TC) was 5.79 mmol/L and the triglyceride (TG) was 4.12 mmol/L. The total cholesterol (TC) was decreased to 5.46 mmol/L and the triglyceride (TG) was decreased to 1.50 mmol/L two weeks after administration of the composition provided by the present invention.

1. A hypolipidemic composition, comprising ornithine, aspartic acid, vitamin B6, vitamin C, and one or more of the following auxiliary components:
citric acid, pantothenic acid, biotin and folic acid;
by a weight ratio:
ornithine:asparticacid:VB6=1:0.1-1.2:0.2-1.5;
ornithine:VC=1:0.05-1.2;
ornithine:citric acid=1:0.001-0.05;
ornithine:pantothenic acid=1:0.00001-0.001;
ornithine:biotin=1:0.000001-0.0005;
ornithine:folic acid=1:0.000001-0.0005.

2. The composition according to claim 1, comprising the following auxiliary components by a weight ratio: asparticacid:VB6=1:0.3-0.7:0.4-0.8.

3. The composition according to claim 2, comprising the following auxiliary components by a weight ratio: asparticacid:VB6=1:0.5:0.6.

4. The composition according to claim 1, comprising the following auxiliary components by a weight ratio: ornithine:VC=1:0.2-0.8.

5. The composition according to claim 4, comprising the following auxiliary components by a weight ratio: ornithine:VC=1:0.5.

6. The composition according to claim 1, comprising the following auxiliary components by a weight ratio: ornithine:citric acid=1:0.001-0.03.

7. The composition according to claim 6, comprising the following auxiliary components by a weight ratio: ornithine:citric acid=1:0.01.

8. The composition according to claim 1, comprising the following auxiliary components by a weight ratio: ornithine:pantothenic acid=1:0.00005-0.0008.

9. The composition according to claim 8, comprising the following auxiliary components by a weight ratio: ornithine:pantothenic acid=1:0.0005.

10. The composition according to claim 1, comprising the following auxiliary components by a weight ratio: ornithine:biotin=1:0.000001-0.0001.

11. The composition according to claim 10, comprising the following auxiliary components by a weight ratio: ornithine:biotin=1:0.000003.

12. The composition according to claim 1, comprising the following auxiliary components by a weight ratio: ornithine:folic acid=1:0.000003-0.0001.

13. The composition according to claim 12, comprising the following auxiliary components by a weight ratio: ornithine:folic acid=1:0.00005.

14. The composition according to claim 1, wherein the ornithine refers to L-ornithine, D-ornithine or DL-ornithine; and the aspartic acid refers to L-aspartic acid, D-aspartic acid or DL-aspartic acid.

15. The composition according to claim 1, wherein the dosage form of the composition as a health-care product or a drug is any one of pharmaceutically acceptable dosage forms

16. The composition according to claim 15, wherein the dosage forms are granules, tablets, capsules, pulvis, pills, powders, medicinal liquors, ointments, pellets, granules, membranes, oral solutions and injections.

17. A method comprising preparing hypolipidemic health-care products, drugs, foods or food additives with the composition according to claim 1.
